(19) United States
(12) Patent Application Publication
(10) Pub. No.: US 2020/0297224 A1
(43) Pub. Date: Sep. 24, 2020

(54) BLOOD PRESSURE ESTIMATION APPARATUS

(71) Applicants: OMRON HEALTHCARE Co., Ltd., Muko-shi (JP); OMRON Corporation, Kyoto-shi (JP)

(72) Inventors: Kentaro MORI, Kyoto (JP); Yasuhiro KAWABATA, Kyoto (JP); Daisuke ISHIHARA, Kyoto (JP)

(73) Assignees: OMRON HEALTHCARE Co., Ltd., Muko-shi (JP); OMRON Corporation, Kyoto-shi (JP)

(21) Appl. No.: 16/899,179

(22) Filed: Jun. 11, 2020

Related U.S. Application Data
(63) Continuation of application No. PCT/JP2018/043356, filed on Nov. 26, 2018.

(30) Foreign Application Priority Data
Dec. 19, 2017 (JP) 2017-242392

(51) Int. Cl. A61B 5/021; A61B 5/00; A61B 5/0235; A61B 5/053

(52) U.S. Cl. CPC A61B 5/02125; A61B 5/681; A61B 2562/0247; A61B 5/0235; A61B 5/053; A61B 5/02141

(57) ABSTRACT

A first fluid bag and a second fluid bag are located side by side along an inner circumference of a belt, expand and contract upon entry and exit of a fluid, and are provided to press a measurement site from therearound while surrounding the measurement site. The fluid supply unit supplies the fluid to the first fluid bag and the second fluid bag. The pulse wave detection unit is disposed on an external surface portion of the first fluid bag and is provided to press the measurement site upon expansion of the first fluid bag. A position of the pulse wave detection unit relative to the artery passing through the measurement site is adjusted through adjustment of a ratio between a volume of the fluid in the first fluid bag and a volume of the fluid in the second fluid bag by the fluid supply unit.

### CROSS REFERENCE TO RELATED APPLICATIONS

The present application is a continuation of International application No. PCT/JP2018/043356, filed Nov. 26, 2018, which claims priority to Japanese Patent Application No. 2017-242392, filed Dec. 19, 2017, the entire contents of each of which are incorporated herein by reference.

### TECHNICAL FIELD

The present invention relates to blood pressure estimation apparatuses, and particularly, to a blood pressure estimation apparatus that estimates a blood pressure based on a pulse transit time.

### BACKGROUND ART

Japanese Patent Laying-Open No. 02-177937 (PTL 1) is a prior art literature disclosing a configuration of a blood pressure monitoring apparatus. The blood pressure monitoring apparatus described in PTL 1 includes a housing shaped into a closed cylinder, a pulse wave sensor, and a pulse wave sensor positioning device. The blood pressure monitoring apparatus is detachably attached to a wrist by a band with an opening end of the housing facing the wrist. The pulse wave sensor and the pulse wave sensor positioning device are provided inside the housing. The pulse wave sensor positioning device includes a pair of rubber bags, an electrically powered pump that supplies a fluid to each of the pair of rubber bags, and a switch valve capable of switching between application of pressure and exhaust of pressure of each of the pair of rubber bags. The pulse wave sensor is disposed between the pair of rubber bags. The pulse wave sensor is positioned relative to a radial artery by controlling the switch valve to adjust the pressure of each of the pair of rubber bags.

Japanese Patent Laying-Open No. 63-275320 (PTL 2) is a prior art literature disclosing a configuration of a pulse wave apparatus. The pulse wave apparatus described in PTL 2 includes a hollow main body with an opening at its lower end, a diaphragm and a contact maker that detect pulse waves of an artery, and moving means for locating the contact maker immediately above the artery. The pulse wave apparatus is detachably attached to a wrist by a band with its opening facing the wrist. The diaphragm, contact maker, and moving means are provided inside the main body. The moving means includes a plurality of bellows and a pressure regulation valve that supplies regulated air to each of the plurality of bellows. The pressure regulation valve is controlled to regulate the pressure of the air to be supplied to each of the plurality of bellows, thereby adjusting the position of the contact maker relative to the artery.

### SUMMARY OF INVENTION

### Technical Problem

The blood pressure monitoring apparatus described in PTL 1 and the pulse wave apparatus described in PTL 2 are each attached to a wrist with the opening end of the housing facing the wrist, and the pulse wave sensor positioning device moves the pulse wave sensor in the housing, thereby adjusting the position of the pulse wave sensor relative to the radial artery. Thus, the range in which the position of the pulse wave sensor is adjustable is limited to the inside of the housing. When a preferable position of the pulse wave sensor is located outside the housing, accordingly, the pulse wave sensor cannot be adjusted to the preferable position.

The present invention has been made in view of the above problem, and an object thereof is to provide a blood pressure estimation apparatus capable of increasing a range in which the position of a pulse wave detection unit of a pulse wave sensor is adjustable, thus stably estimating a blood pressure.

### Solution to Problem

A blood pressure estimation apparatus according to the present invention includes a belt, a first fluid bag and a second fluid bag, a pulse wave sensor, a fluid supply unit, a first pressure sensor, and a second pressure sensor. The belt surrounds a measurement site. The first fluid bag and the second fluid bag are located side by side along an inner circumference of the belt, expand and contract upon entry and exit of a fluid, and are provided to press the measurement site from therearound while surrounding the measurement site. The pulse wave sensor includes a pulse wave detection unit that detects a pulse wave of an artery passing through the measurement site. The fluid supply unit supplies the fluid to the first fluid bag and the second fluid bag. The first pressure sensor detects a pressure in the first fluid bag. The second pressure sensor detects a pressure in the second fluid bag. The pulse wave detection unit is disposed on an external surface portion of the first fluid bag and provided to press the measurement site upon expansion of the first fluid bag. A position of the pulse wave detection unit relative to the artery passing through the measurement site is adjusted through adjustment of a ratio between a volume of the fluid in the first fluid bag and a volume of the fluid in the second fluid bag by the fluid supply unit.

In one embodiment of the present invention, the pulse wave detection unit detects a pulse wave based on a change in an impedance of the artery passing through the measurement site.

In one embodiment of the present invention, the fluid supply unit includes a pump that delivers the fluid, a first on-off valve connected between the first fluid bag and the pump, and a second on-off valve connected between the second fluid bag and the pump.

### Advantageous Effects of Invention

The present invention can increase the range in which the position of the pulse wave detection unit of the pulse wave sensor is adjustable, thus stably estimating a blood pressure.

### BRIEF DESCRIPTION OF DRAWINGS

FIG. 1 is a perspective view showing an appearance of a blood pressure estimation apparatus according to an embodiment of the present invention.

FIG. 2 is a sectional view showing a state in which the blood pressure estimation apparatus according to the embodiment of the present invention is attached to a measurement site.

FIG. 3 shows an arrangement of a pulse wave detection unit of a pulse wave sensor with the blood pressure estimation apparatus according to the embodiment of the present invention being attached to the measurement site.

FIG. 4 is a block diagram showing a configuration of the blood pressure estimation apparatus according to the embodiment of the present invention.

FIG. 5 is a sectional view showing how the blood pressure estimation apparatus according to the embodiment of the present invention, which is attached to the measurement site, measures a blood pressure by the oscillometric method.

FIG. 6A is a sectional view showing how the blood pressure estimation apparatus according to the embodiment of the present invention, which is attached to the measurement site, measures blood pressure propagation times, and FIG. 6B shows pulse transit times of a radial artery detected by a first pulse wave detection unit and a second pulse wave detection unit of the blood pressure estimation apparatus according to the embodiment of the present invention.

FIG. 7 is a graph showing experimental results of the calculation of a cross-correlation coefficient between a pulse wave signal detected by the first pulse wave detection unit and a pulse wave signal detected by the second pulse wave detection unit by changing strengths of pressing the first pulse wave detection unit and the second pulse wave detection unit against a palm lateral surface of a left wrist.

FIG. 8 is a sectional view showing a state in which a ratio between a fluid volume in the first fluid bag and a fluid volume in the second fluid bag is adjusted in the blood pressure estimation apparatus according to the embodiment of the present invention.

FIG. 9 is a flowchart showing an operation flow in estimation of a blood pressure by the blood pressure estimation apparatus according to the embodiment of the present invention based on a pulse transit time.

### DESCRIPTION OF EMBODIMENTS

A blood pressure estimation apparatus according to an embodiment of the present invention will now be described with reference to the drawings, in which the same or corresponding parts are designated by the same reference numerals, and description thereof will not be repeated.

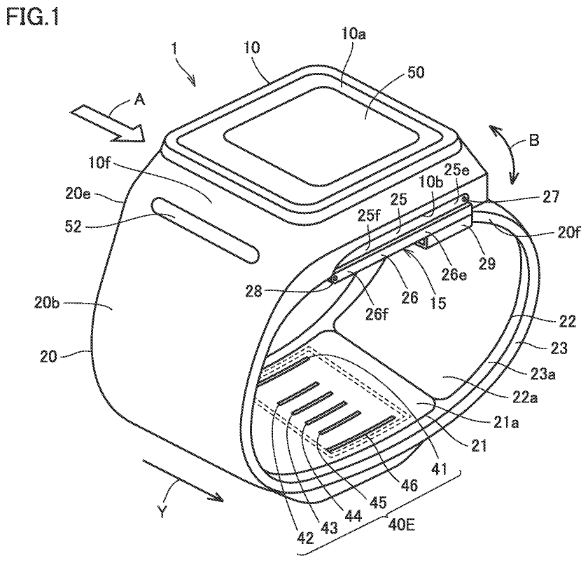

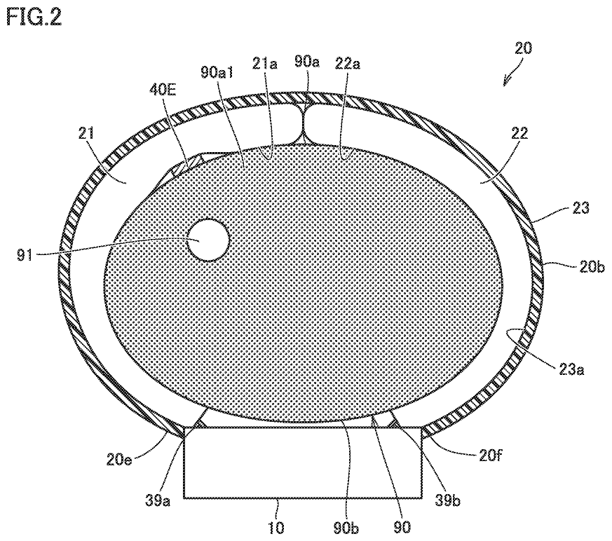

FIG. 1 is a perspective view showing an appearance of a blood pressure estimation apparatus according to an embodiment of the present invention. FIG. 2 is a sectional view showing a state in which the blood pressure estimation apparatus according to the embodiment of the present invention is attached to a measurement site. FIG. 2 shows a cross-section perpendicular to the longitudinal direction of a left wrist. In the present embodiment, the measurement site is the left wrist. The measurement site may be a right wrist.

As shown in FIGS. 1 and 2, a blood pressure estimation apparatus 1 according to an embodiment of the present invention includes a display unit 10, a belt portion 20, and a pulse wave sensor. Display unit 10 displays the result of blood pressure estimation of blood pressure estimation apparatus 1. Belt portion 20 is connected to display unit 10 and surrounds a left wrist 90, which is a measurement site. The pulse wave sensor includes a pulse wave detection unit 40E, which detects a pulse wave of an artery passing through the measurement site.

Blood pressure estimation apparatus 1 is mainly composed of belt portion 20 surrounding left wrist 90, which is the measurement site, and display unit 10 connected to belt portion 20.

As shown in FIG. 1, display unit 10 has an outer shape of a truncated quadrangular pyramid which projects outwardly from belt portion 20. Display unit 10 preferably has a small size and a low profile so as not to hinder activities of a subject.

Display unit 10 is provided with a display 50 and an operation unit 52. Display 50 is disposed on a top surface portion 10a of display unit 10. Operation unit 52 is disposed on a lateral surface portion 10f of display unit 10.

Display unit 10 is provided integrally with one end 20e of belt portion 20 through integral molding. In another configuration, belt portion 20 and display unit 10 may be formed separately and connected to each other by, for example, an engaging member such as a hinge. As shown in FIG. 1, a bottom surface 10b of display unit 10 and an end 20f of belt portion 20 are connected to each other by a buckle 15.

Buckle 15 includes a plate-shaped member 25, which is disposed on the outer circumferential side, and a plate-shaped member 26, which is disposed on the inner circumferential side. One end 25e of plate-shaped member 25 is pivotally attached to display unit 10 via a coupling rod 27 running in a width direction Y. The other end 25f of plate-shaped member 25 is pivotally attached to the other end 26f of plate-shaped member 26 via a coupling rod 28 running in width direction Y. One end 26e of plate-shaped member 26 is fixed to the vicinity of end 20f of belt portion 20 by a fixing portion 29.

For the circumferential direction of belt portion 20, the position of attachment of fixing portion 29 is adjusted in advance in accordance with the circumferential length of left wrist 90 of a subject. Blood pressure estimation apparatus 1 has a substantially annular shape in its entirety. A portion between bottom surface 10b of display unit 10 and end 20f of belt portion 20 is configured to be opened/closed by buckle 15 in the direction of arrow B in FIG. 1.

Belt portion 20 includes a belt 23, and a first fluid bag 21 and a second fluid bag 22, which are provided on the inner circumferential side of belt 23 and are capable of expanding and contracting. The dimension of belt portion 20 in width direction Y is, for example, approximately 30 mm. Belt 23 is an elongated band-shaped member surrounding left wrist 90 in the circumferential direction. Belt 23 has an outer circumferential portion 20b. Belt 23 is formed of a plastic material that is flexible in the thickness direction and is not elastic in the circumferential direction.

First fluid bag 21 and second fluid bag 22 are attached to belt 23. First fluid bag 21 and second fluid bag 22 are positioned side by side on an inner circumferential portion 23a of belt 23. The inner circumferential portion of belt portion 20 which contacts left wrist 90 is composed of a first inner circumferential portion 21a and a second inner circumferential portion 22a. First fluid bag 21 has an external surface portion forming first inner circumferential portion 21a. Second fluid bag 22 has an external surface portion forming second inner circumferential portion 22a.

Each of first fluid bag 21 and second fluid bag 22 is formed through welding of the circumferential portions of two stretchable polyurethane sheets overlaid with each other, thus being shaped into a bag capable of receiving a fluid. The fluid includes both of liquid and gas, and for example, water, air, or the like can be used as the fluid. First fluid bag 21 and second fluid bag 22 expand and contract upon entry and exit of the fluid and are provided to press left wrist 90 from therearound while surrounding left wrist 90.

Blood pressure estimation apparatus 1 is provided with a fluid supply unit that supplies a fluid to first fluid bag 21 and second fluid bag 22. Blood pressure estimation apparatus 1 is provided with a first pressure sensor that detects a pressure in first fluid bag 21 and a second pressure sensor that detects a pressure in second fluid bag 22.

A pulse wave detection unit 40E of the pulse wave sensor is provided on first inner circumferential portion 21a of belt portion 20. In the present embodiment, pulse wave detection unit 40E of the pulse wave sensor is provided on the external surface portion of first fluid bag 21 of first inner circumferential portion 21a of belt portion 20. Pulse wave detection unit 40E is provided to press left wrist 90 upon expansion of first fluid bag 21.

Pulse wave detection unit 40E of the pulse wave sensor is composed of six electrodes spaced from each other in width direction Y of belt portion 20. Specifically, a current electrode 41, a detection electrode 42, a detection electrode 43, a detection electrode 44, a detection electrode 45, and a current electrode 46 are arranged side by side in a row in order from one side of width direction Y. Detection electrode 42 and detection electrode 43 constitute a first pulse wave detection unit. Detection electrode 44 and detection electrode 45 constitute a second pulse wave detection unit.

Each of a spacing between detection electrode 42 and detection electrode 43 and a spacing between detection electrode 44 and detection electrode 45 in width direction Y of belt portion 20 is, for example, 2 mm. Each of current electrode 41, detection electrode 42, detection electrode 43, detection electrode 44, detection electrode 45, and current electrode 46 has a rectangular outer shape and is formed with a low profile and flexibility.

With blood pressure estimation apparatus 1 attached to left wrist 90, pulse wave detection unit 40E is disposed corresponding to a radial artery 91 of left wrist 90. Radial artery 91 passes through the vicinity of a palm lateral surface 90a of left wrist 90, which is the surface on the palm side, within left wrist 90. In the present embodiment, pulse wave detection unit 40E detects a pulse wave based on a change in the impedance of radial artery 91 passing through left wrist 90.

The method of detecting pulse waves by the pulse wave detection unit is not limited to the method of detecting a pulse wave based on changes in the impedance of an artery. For example, the pulse wave sensor may include a light emitting element that radiates light toward an artery passing through a corresponding portion of the measurement site and a right receiving element that receives reflected light or transmitted light of the light and detect a change in the volume of the artery as a pulse wave.

The pulse wave sensor may include a piezoelectric sensor held in contact with the measurement site and detect a distortion due to a pressure of the artery passing through a corresponding portion of the measurement site as a change in electrical resistance. Further, the pulse wave sensor may include a transmission element that transmits a radio wave toward an artery passing through a corresponding portion of the measurement site and a reception element that receives a reflected wave of the electric wave and detect a change in the distance between the artery and the sensor due to a pulse wave of the artery as a phase deviation between a transmission wave and a reflective wave.

In attachment of blood pressure estimation apparatus 1 to left wrist 90, the subject passes the left hand through belt portion 20 from the direction indicated by arrow A in FIG. 1 with buckle 15 being opened for an increased annular diameter of belt portion 20. Then, as shown in FIG. 2, the subject adjusts the angular position of belt portion 20 around left wrist 90 to position pulse wave detection unit 40E of the pulse wave sensor such that pulse wave detection unit 40E faces radial artery 91 passing through left wrist 90.

Thus, pulse wave detection unit 40E of the pulse wave sensor is held in contact with a portion 90a1 of palm lateral surface 90a of left wrist 90, which corresponds to radial artery 91. In this state, the subject closes and fixes buckle 15. Consequently, the subject attaches blood pressure estimation apparatus 1 onto left wrist 90. With blood pressure estimation apparatus 1 attached to left wrist 90, display unit 10 is disposed corresponding to a rear surface 90b of left wrist 90, which is the surface on the back side of the hand.

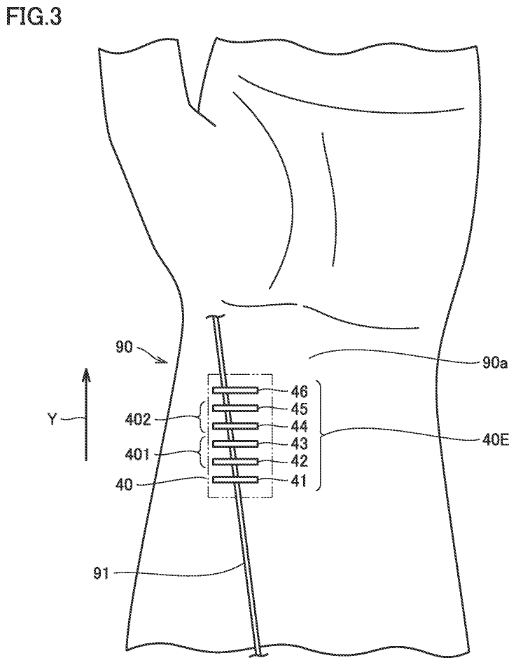

FIG. 3 shows the arrangement of the pulse wave detection unit of the pulse wave sensor with the blood pressure estimation apparatus according to the embodiment of the present invention being attached to the measurement site. As shown in FIG. 3, with blood pressure estimation apparatus 1 attached to left wrist 90, pulse wave detection unit 40E of the pulse wave sensor is preferably located along radial artery 91.

A second pulse wave detection unit 402 composed of detection electrode 44 and detection electrode 45 is disposed downstream of a first pulse wave detection unit 401 composed of detection electrode 42 and detection electrode 43 in a bloodstream of radial artery 91. A spacing between first pulse wave detection unit 401 and second pulse wave detection unit 402 in width direction Y of belt portion 20 is, for example, 20 mm. In other words, a distance between a midpoint between detection electrode 42 and detection electrode 43 and a midpoint between detection electrode 44 and detection electrode 45 in width direction Y of belt portion 20 is, for example, 20 mm.

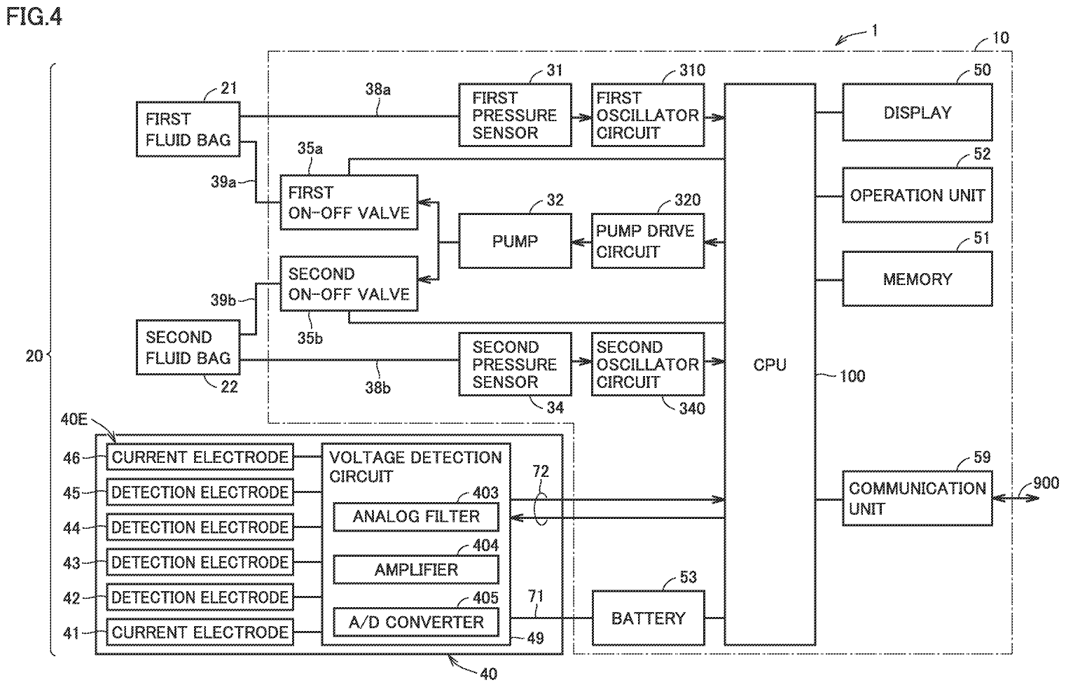

The components of blood pressure estimation apparatus 1 will now be described in detail. FIG. 4 is a block diagram showing a configuration of the blood pressure estimation apparatus according to the embodiment of the present invention.

As shown in FIG. 4, display unit 10 is provided with a central processing unit (CPU) 100, display 50, a memory 51, operation unit 52, a battery 53, and a communication unit 59.

Display unit 10 is also provided with a first pressure sensor 31, a second pressure sensor 34, a pump 32, a first on-off valve 35a, and a second on-off valve 35b. Pump 32 delivers a fluid to first fluid bag 21 and second fluid bag 22. First on-off valve 35a is connected between first fluid bag 21 and pump 32. Second on-off valve 35b is connected between second fluid bag 22 and pump 32.

Further, display unit 10 is provided with a first oscillator circuit 310, which converts an output of first pressure sensor 31 to a frequency, a second oscillator circuit 340, which converts an output of second pressure sensor 34 to a frequency, and a pump drive circuit 320, which drives pump 32.

Pulse wave sensor 40 includes pulse wave detection unit 40E and a current feed and voltage detection circuit 49. Each of current electrode 41, detection electrode 42, detection electrode 43, detection electrode 44, detection electrode 45, and current electrode 46 is connected with current feed and voltage detection circuit 49. Current feed and voltage detection circuit 49 is connected with CPU 100 through a signal wire 72.

Display 50 is implemented by, for example, an organic electro luminescence (EL) display, and displays information on estimation of a blood pressure, such as a blood pressure estimation result, and any other information in response to a control signal from CPU 100. Display 50 is not limited to the organic EL display and may be implemented by any other type of display, such as a liquid crystal display (LCD).

Operation unit 52 is implemented by, for example, a push switch, and provides CPU 100 with an operation signal corresponding to an instruction to start or stop the estimation of a blood pressure by a subject. Operation unit 52 is not limited to the push switch and may be implemented by, for example, a touch panel switch, such as a pressure-sensitive switch or a proximity touch panel switch. Alternatively, display unit 10 may be provided with a microphone, and an instruction to start or stop the estimation of a blood pressure by voice of the subject may be provided to CPU 100 through the microphone.

Memory 51 stores in a non-transitory manner a program for controlling blood pressure estimation apparatus 1, data used for controlling blood pressure estimation apparatus 1, setting data for setting various functions of blood pressure estimation apparatus 1, and data on the results of blood pressure estimation. Memory 51 is also used as a work memory in execution of a program.

CPU 100 controls various functions of blood pressure estimation apparatus 1 in accordance with the program for controlling blood pressure estimation apparatus 1 stored in memory 51. For example, when a blood pressure is measured by the oscillometric method, CPU 100 drives pump 32 based on signals from first pressure sensor 31 and second pressure sensor 34 in response to the instruction to start the measurement of a blood pressure from operation unit 52, thereby rendering first on-off valve 35a and second on-off valve 35b open. CPU 100 calculates a blood pressure based on the signals from first pressure sensor 31 and second pressure sensor 34.

When estimating a blood pressure based on a pulse transit time, CPU 100 drives pump 32 based on the signals from first pressure sensor 31 and second pressure sensor 34 in response to the instruction to start the estimation of a blood pressure from operation unit 52, thereby controlling the open/closed state of first on-off valve 35a and second on-off valve 35b.

Communication unit 59 is controlled by CPU 100 to transmit predetermined information to an external device through a network 900 or communicate the information received from the external device through network 900 to CPU 100. The communications performed by network 900 may be either wireless communications or wired communications. For example, network 900 is the Internet, which is not limited thereto. Network 900 may be any other type of network, such as local area network (LAN), or one-to-one communication using a USB cable or the like. Communication unit 59 may include a micro-USB connector.

Pump 32 and first on-off valve 35a are connected to first fluid bag 21 through a first air pipe 39a. Pump 32 and second on-off valve 35b are connected to second fluid bag 22 through a second air pipe 39b. Pump 32 is, for example, a piezoelectric pump. Pump 32 supplies air into first fluid bag 21 through first air pipe 39a in order to pressurize first fluid bag 21. Pump 32 supplies air into second fluid bag 22 through second air pipe 39b in order to pressurize second fluid bag 22.

First pressure sensor 31 is connected to first fluid bag 21 through a first air pipe 38a. First pressure sensor 31 detects the pressure in first fluid bag 21 through first air pipe 38a. First pressure sensor 31 is, for example, a piezoresistive pressure sensor. For example, first pressure sensor 31 outputs, as time-series signals, pressures detected with atmospheric pressure being defined as a zero point.

Similarly, second pressure sensor 34 is connected to second fluid bag 22 through a second air pipe 38b. Second pressure sensor 34 detects the pressure in second fluid bag 22 through second air pipe 38b. Second pressure sensor 34 is, for example, a piezoresistive pressure sensor. For example, second pressure sensor 34 outputs, as time-series signals, pressures detected with atmospheric pressure being defined as a zero point.

Each of first on-off valve 35a and second on-off valve 35b operates to be opened and closed based on a control signal supplied from CPU 100. Pump drive circuit 320 drives pump 32 based on a control signal supplied from CPU 100.

First oscillator circuit 310 outputs, to CPU 100, a frequency signal with a frequency corresponding to an electrical signal value which is based on a change in the electrical resistance due to the piezo resistance effect from first pressure sensor 31. The output of first pressure sensor 31 is used to control the pressure in first fluid bag 21 and to calculate a blood pressure by the oscillometric method.

Similarly, second oscillator circuit 340 outputs, to CPU 100, a frequency signal with a frequency corresponding to an electrical signal value which is based on a change in the electrical resistance due to the piezo resistance effect from second pressure sensor 34. The output of second pressure sensor 34 is used to control the pressure in second fluid bag 22 and to calculate a blood pressure by the oscillometric method.

Blood pressures calculated by the oscillometric method include a systolic blood pressure (SBP) and a diastolic blood pressure (DBP).

Battery 53 supplies electric power to various elements mounted on display unit 10. Battery 53 also supplies electric power to current feed and voltage detection circuit 49 of pulse wave sensor 40 through a line 71. Line 71 is provided to extend between display unit 10 and pulse wave sensor 40 in the circumferential direction of belt portion 20 while being sandwiched between belt 23 and first fluid bag 21 of belt portion 20 together with signal wire 72. Battery 53 is also connected with CPU 100.

Voltage detection circuit 49 of pulse wave sensor 40 operates based on a control signal supplied from CPU 100. Specifically, voltage detection circuit 49 includes an analog filter 403, an amplifier 404, and an analog/digital (A/D) converter 405. Voltage detection circuit 49 may further include a step-up circuit that boosts a power supply voltage and a voltage regulation circuit that regulates the boosted voltage to a predetermined voltage.

Following will describe an operation of blood pressure estimation apparatus 1 in estimation of a blood pressure with blood pressure estimation apparatus 1 according to the embodiment of the present invention.

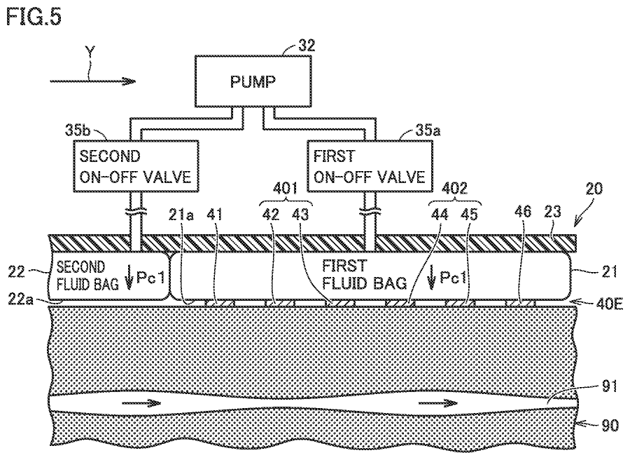

First, blood pressure estimation apparatus 1 measures a blood pressure by the oscillometric method. FIG. 5 is a sectional view showing how the blood pressure estimation apparatus according to the embodiment of the present invention, which is attached to a measurement site, measures a blood pressure by the oscillometric method. FIG. 5 shows a cross-section taken in the longitudinal direction of the left wrist.

Upon receipt of an instruction to start the measurement of a blood pressure from operation unit 52, CPU 100 of blood pressure estimation apparatus 1 renders first on-off valve 35a and second on-off valve 35b open and drives pump 32 through pump drive circuit 320, thereby supplying air into first fluid bag 21 and second fluid bag 22. This expands first fluid bag 21 and second fluid bag 22 and gradually pressurizes first fluid bag 21 and second fluid bag 22. As shown in FIG. 5, first fluid bag 21 and second fluid bag 22 extend in the circumferential direction of left wrist 90, and are pressurized by pump 32 to press the circumference of left wrist 90 uniformly at a pressure Pc1.

During pressurization, in order to calculate a blood pressure, CPU 100 monitors pressure Pc1 in first fluid bag 21 with first pressure sensor 31 and also pressure Pc1 in second fluid bag 22 with second pressure sensor 34 and obtains a fluctuation component of the volume of the artery, which occurs in radial artery 91 of left wrist 90, as a pulse wave signal. CPU 100 is not necessarily required to obtain a pulse wave signal based on both of pressure Pc1 in first fluid bag 21 and pressure Pc1 in second fluid bag 22 and may be only required to obtain a pulse wave signal based on at least one of pressure Pc1 in first fluid bag 21 and pressure Pc1 in second fluid bag 22.

Based on the obtained pulse wave signal, CPU 100 applies a publicly known algorithm by the oscillometric method and begins to calculate each of a systolic blood pressure and a diastolic blood pressure. When CPU 100 has not yet calculated a blood pressure due to lack of data, unless pressure Pc1 in first fluid bag 21 and pressure Pc1 in second fluid bag 22 have not reached an upper limit pressure, for example, approximately 300 mmHg, CPU 100 begins to boost pressure Pc1 in first fluid bag 21 and pressure Pc1 in second fluid bag 22 further and calculate a blood pressure again.

When CPU 100 has successfully calculated a blood pressure, CPU 100 stops pump 32 through pump drive circuit 320. CPU 100 displays the result of blood pressure measurement on display 50 and also records the result in memory 51. A blood pressure may be calculated not only during pressurization but also during decompression.

Since only pulse wave detection unit 40E is located between the external surface portion of first fluid bag 21 of first inner circumferential portion 21a of belt portion 20 and left wrist 90, pressing by first fluid bag 21 is not hindered by any other member, so that a blood vessel can be closed sufficiently. Since any other member is not located between the external surface portion of second fluid bag 22 of second inner circumferential portion 22a of belt portion 20 and left wrist 90, pressing by second fluid bag 22 is not hindered by the other member, so that a blood vessel can be closed sufficiently. A blood pressure can thus be measured by the oscillometric method with high accuracy.

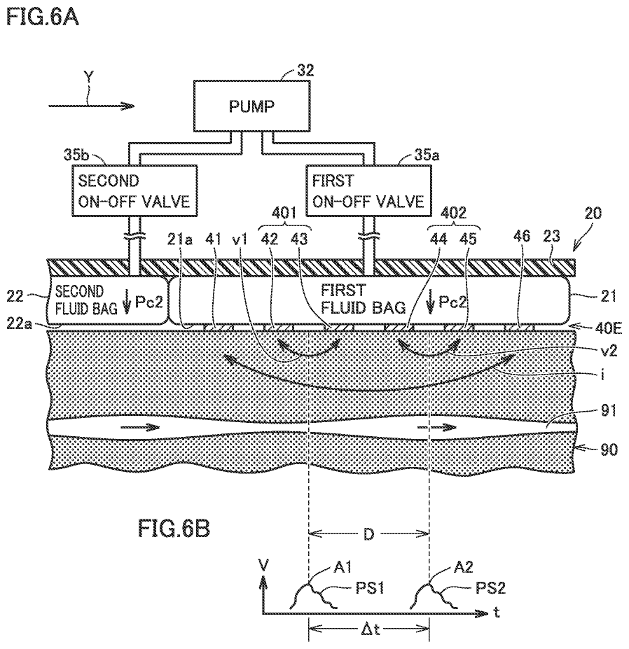

Blood pressure estimation apparatus 1 then measures a pulse transit time. FIG. 6A is a sectional view showing how the blood pressure estimation apparatus according to the embodiment of the present invention, which is attached to the measurement site, measures blood pressure propagation times, and FIG. 6B shows pulse transit times of a radial artery which are detected by the first pulse wave detection unit and the second pulse wave detection unit of the blood pressure estimation apparatus according to the embodiment of the present invention. FIG. 6A shows a cross-section taken in the longitudinal direction of the left wrist. In FIG. 6B, the vertical axis represents voltage (V), and the horizontal axis represents time.

First, in detection of a pulse wave of radial artery 91, CPU 100 of blood pressure estimation apparatus 1 renders first on-off valve 35a and second on-off valve 35b open and drives pump 32 through pump drive circuit 320, thereby supplying air into first fluid bag 21 and second fluid bag 22.

Consequently, first fluid bag 21 and second fluid bag 22 are expanded, and first fluid bag 21 and second fluid bag 22 are gradually pressurized. Each of first fluid bag 21 and second fluid bag 22 is pressurized by pump 32, so that the inner pressure attains to Pc2 lower than Pc1, as shown in FIG. 6A. Each of first pulse wave detection unit 401 and second pulse wave detection unit 402 is pressed against palm lateral surface 90a of left wrist 90 through expansion of first fluid bag 21. Specifically, upon receipt of a pressing force corresponding to pressure Pc2 in first fluid bag 21, each of first pulse wave detection unit 401 and second pulse wave detection unit 402 is pressed against palm lateral surface 90a of left wrist 90.

In order to detect a pulse wave of a radial artery, current feed and voltage detection circuit 49 applies a voltage between current electrode 41 and current electrode 46 to flow a current i, which has, for example, a frequency of 50 kHz and a current value of 1 mA. In this state, current feed and voltage detection circuit 49 detects a voltage signal v1 between detection electrode 42 and detection electrode 43 and a voltage signal v2 between detection electrode 44 and detection electrode 45.

Specifically, current feed and voltage detection circuit 49 accepts an input of voltage signal v1 detected by first pulse wave detection unit 401 and accepts an input of voltage signal v2 detected by second pulse wave detection unit 402.

Voltage signal v1 represents a change in the electrical impedance in a portion of palm lateral surface 90a of left wrist 90, which faces first pulse wave detection unit 401, due to a pulse wave of a bloodstream of radial artery 91. Voltage signal v2 represents a change in the electrical impedance in a portion of palm lateral surface 90a of left wrist 90, which faces second pulse wave detection unit 402, due to a pulse wave of a bloodstream of radial artery 91.

Analog filter 403 of current feed and voltage detection circuit 49 has a transfer function G and performs filtering on the amplified voltage signal v1 and voltage signal v2. Specifically, analog filter 403 removes noise of other than frequencies that characterize voltage signal v1 and voltage signal v2 and performs filtering for improving a signal-noise ratio (SN ratio). Amplifier 404 is implemented by, for example, an operational amplifier and amplifies the filtered voltage signal v1 and voltage signal v2. A/D converter 405 converts the amplified voltage signal v1 and voltage signal v2 from analog data to digital data and outputs the digital data to CPU 100 through line 72.

CPU 100 performs signal processing on digital data of each of the received voltage signal v1 and voltage signal v2, thereby generating a pulse wave signal PS1 and a pulse wave signal PS2 each having a waveform with a crest as shown in FIG. 6B. CPU 100 further calculates a time difference Δt between a peak A1 of pulse wave signal PS1 and a peak A2 of pulse wave signal PS2. Time difference Δt is a pulse transit time (PTT).

The voltage value of each of voltage signal v1 and voltage signal v2 is, for example, approximately 1 my. Each of peak A1 of pulse wave signal PS1 and peak A2 of pulse wave signal PS2 is, for example, approximately 1 V. Assuming that a pulse wave velocity (PWV) of a bloodstream of radial artery 91 is in the range of 1000 cm/s or more and 2000 cm/s or less, when a distance D between first pulse wave detection unit 401 and second pulse wave detection unit 402 is 20 mm, a time difference Δt between pulse wave signal PS1 and pulse wave signal PS2 is in the range of 1.0 ms or more and 2.0 ms or less.

CPU 100 performs calibration between a blood pressure measured by the oscillometric method and a pulse transit time Δt to associate the blood pressure and pulse transit time Δt with each other. As a result, a blood pressure can be estimated based on pulse transit time Δt.

In the estimation of a blood pressure based on pulse transit time Δt, the cross-correlation coefficient between pulse wave signal PS1 and pulse wave signal PS2 should exceed a threshold for guaranteeing reliability.

Description will now be given of an example experiment in which the cross-correlation coefficient between pulse wave signal PS1 and pulse wave signal PS2 was calculated by changing the force of pressing of pulse wave detection unit 40E against palm lateral surface 90a of left wrist 90.

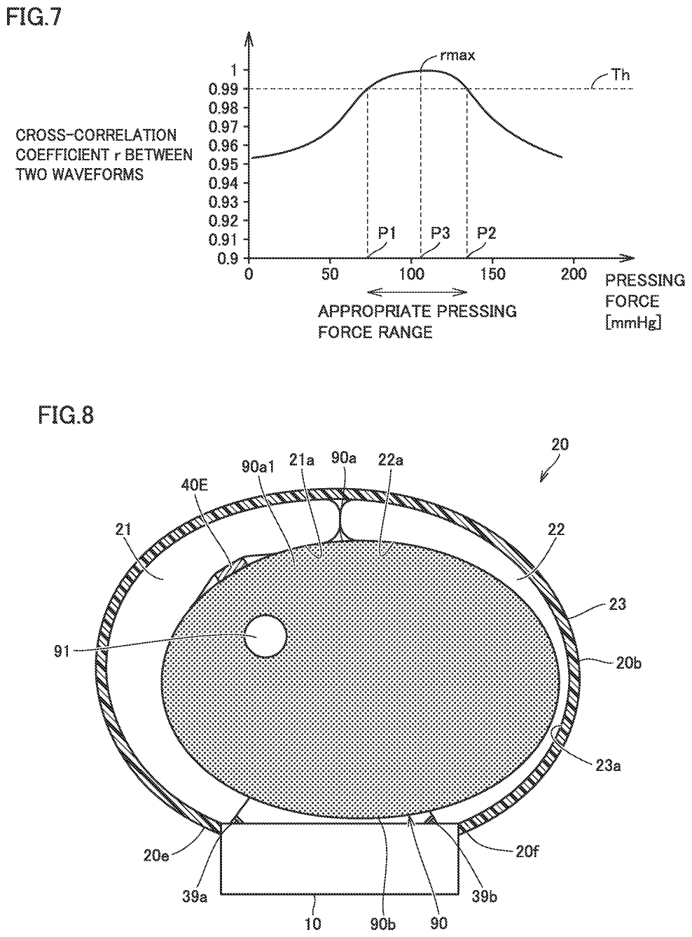

FIG. 7 is a graph showing experimental results of the calculation of a cross-correlation coefficient between a pulse wave signal detected by the first pulse wave detection unit and a pulse wave signal detected by the second pulse wave detection unit by changing the force of pressing of the first pulse wave detection unit and the second pulse wave detection unit against the palm lateral surface of the left wrist. In FIG. 7, the vertical axis represents a cross-correlation coefficient r between two waveforms of pulse wave signal PS1 and pulse wave signal PS2, and the horizontal axis represents a force (mmHg) of pressing of the first pulse wave detection unit and the second pulse wave detection unit against the palm lateral surface of the left wrist.

In this example experiment, cross-correlation coefficient r between pulse wave signal PS1 and pulse wave signal PS2 was calculated while gradually increasing pressure Pc2 in first fluid bag 21, which is the force of pressing of each of first pulse wave detection unit 401 and second pulse wave detection unit 402 against palm lateral surface 90a of left wrist 90, from 0 mmHg. A threshold Th of cross-correlation coefficient r was set to 0.99.

As shown in FIG. 7, as the pressing force increased from 0 mmHg, cross-correlation coefficient r increased to a maximum value r max, and then decreased after reaching maximum value r max. In the range of pressing force of 72 mmHg or more and 150 mmHg or less, cross-correlation coefficient r exceeded threshold Th. This range is an appropriate pressing force range. That is to say, the appropriate pressing force range has a lower limit P1 of 72 mmHg and an upper limit P2 of 150 mmHg. When the value of pressing force was P3 within the appropriate pressing force range, cross-correlation coefficient r had maximum value r max.

Although cross-correlation coefficient r between pulse wave signal PS1 detected by first pulse wave detection unit 401 and pulse wave signal PS2 detected by second pulse wave detection unit 402 was calculated by changing the force of pressing of first pulse wave detection unit 401 and second pulse wave detection unit 402 against palm lateral surface 90a of left wrist 90 in this example experiment, even when the pressing force is uniform, cross-correlation coefficient r fluctuates as the positions of first pulse wave detection unit 401 and second pulse wave detection unit 402 change relative to radial artery 91 of left wrist 90.

Specifically, there is an appropriate position range of each of first pulse wave detection unit 401 and second pulse wave detection unit 402 relative to radial artery 91 of left wrist 90. When at least one of first pulse wave detection unit 401 and second pulse wave detection unit 402 is located outside of this appropriate position range, cross-correlation coefficient r is equal to or less than threshold Th, resulting in reduced reliability of a blood pressure estimate.

In blood pressure estimation apparatus 1 according to the present embodiment, then, when cross-correlation coefficient r is less than or equal to threshold Th even though the pressing force is within the appropriate pressing force range, CPU 100 determines that at least one of first pulse wave detection unit 401 and second pulse wave detection unit 402 is located outside of the appropriate position range and causes the fluid supply unit to adjust the ratio between the volume of the fluid in first fluid bag 21 and the volume of the fluid in second fluid bag 22, thereby adjusting the positions of first pulse wave detection unit 401 and second pulse wave detection unit 402 relative to radial artery 91 of left wrist 90.

FIG. 8 is a sectional view showing a state in which a ratio between a fluid volume in the first fluid bag and a fluid volume in the second fluid bag is adjusted in the blood pressure estimation apparatus according to the embodiment of the present invention. FIG. 8 shows a cross-section perpendicular to the longitudinal direction of the left wrist.

As shown in FIG. 8, as the volume of the fluid in first fluid bag 21 is increased and the volume of the fluid in second fluid bag 22 is decreased, left wrist 90 is pressed by first fluid bag 21 to move toward second fluid bag 22 in the range surrounded by belt 23. This changes the positions of first pulse wave detection unit 401 and second pulse wave detection unit 402 relative to radial artery 91 of left wrist 90.

CPU 100 calculates cross-correlation coefficient r in this state and, when cross-correlation coefficient r exceeds threshold Th, determines that first pulse wave detection unit 401 and second pulse wave detection unit 402 are located within the appropriate position range relative to radial artery 91 of left wrist 90.

Conversely, when cross-correlation coefficient r becomes further apart from threshold Th, as the volume of the fluid in first fluid bag 21 is decreased and the volume of the fluid in second fluid bag 22 is increased, left wrist 90 is pressed by second fluid bag 22 to move toward first fluid bag 21 in the range surrounded by belt 23. CPU 100 calculates cross-correlation coefficient r in this state and, until cross-correlation coefficient r exceeds threshold Th, repeatedly adjusts the positions of first pulse wave detection unit 401 and second pulse wave detection unit 402 relative to radial artery 91 of left wrist 90.

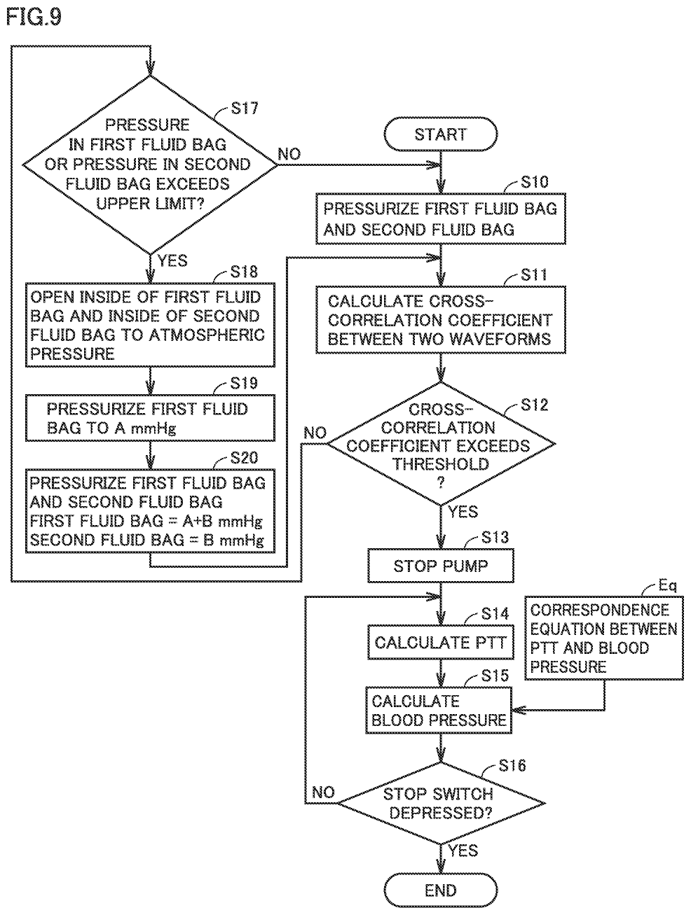

Description will now be given of an operation flow in the estimation of a blood pressure based on pulse transit time Δt by blood pressure estimation apparatus 1 according to the embodiment of the present invention. FIG. 9 is a flowchart showing an operation flow in estimation of a blood pressure by the blood pressure estimation apparatus according to the embodiment of the present invention based on pulse transit time estimates.

As shown in FIG. 9, CPU 100 of blood pressure estimation apparatus 1 according to the embodiment of the present invention pressurizes first fluid bag 21 and second fluid bag 22 (S10). CPU 100 then calculates cross-correlation coefficient r between pulse wave signal PS1 detected by first pulse wave detection unit 401 and pulse wave signal PS2 detected by second pulse wave detection unit 402 in real time (S11).

CPU 100 then determines whether cross-correlation coefficient r exceeds threshold Th (S12). When cross-correlation coefficient r is less than or equal to threshold Th, CPU 100 determines whether the pressure in first fluid bag 21 or the pressure in second fluid bag 22 exceeds the upper limit (S17). This upper limit is set to such a pressure that would not put an enormous burden on a subject.

When the pressure in first fluid bag 21 and the pressure in second fluid bag 22 do not exceed the upper limit, CPU 100 repeats the processes of steps S10 to S12 in order to set the force of pressing of first pulse wave detection unit 401 and second pulse wave detection unit 402 against palm lateral surface 90a of left wrist 90 within the appropriate pressing force range.

When the pressure in first fluid bag 21 or the pressure in second fluid bag 22 exceeds the upper limit, CPU 100 determines that at least one of first pulse wave detection unit 401 and second pulse wave detection unit 402 is located outside of the appropriate position range, and temporarily opens the inside of first fluid bag 21 and the inside of second fluid bag 22 to atmospheric pressure (S18). Specifically, CPU 100 opens first on-off valve 35a and second on-off valve 35b with pump 32 being stopped.

CPU 100 then pressurizes first fluid bag 21 to, for example, A mmHg (S19). Specifically, CPU 100 opens first on-off valve 35a and closes second on-off valve 35b with pump 32 being driven.

CPU 100 then pressurizes first fluid bag 21 and second fluid bag 22 (S20). For example, CPU 100 pressurizes first fluid bag 21 to A+B mmHg and pressurizes second fluid bag 22 to B mmHg. Specifically, CPU 100 opens first on-off valve 35a and second on-off valve 35b with pump 32 being driven.

CPU 100 performs the processes of S11 to S12 again in order to check whether first pulse wave detection unit 401 and second pulse wave detection unit 402, the positions of which have been adjusted, are located within the appropriate position range.

When cross-correlation coefficient r exceeds threshold Th, CPU 100 stops pump 32 (S13). In this state, CPU 100 calculates, as pulse transit time (PTT), time difference Δt between peak A1 of pulse wave signal PS1 and peak A2 of pulse wave signal PS2 (S14).

CPU 100 then calculates and estimates a blood pressure based on pulse transit time Δt using a correspondence equation Eq between pulse transit time Δt and blood pressure associated with each other through calibration (S15). Correlation equation Eq may be a publicly known fractional function.

CPU 100 then checks whether an instruction to stop measurement has been provided from operation unit 52 (S16). When the instruction to stop measurement has not been provided from operation unit 52, CPU 100 periodically repeats the calculation of pulse transit time Δt (S14) and the estimation of blood pressure (S15) every time pulse wave signal PS1 and pulse wave signal PS2 are provided in accordance with a pulse wave. CPU 100 displays the result of blood pressure estimation on display 50 and records the result in memory 51. Upon receipt of the instruction to stop measurement from operation unit 52, CPU 100 ends the blood pressure estimation operation.

In blood pressure estimation apparatus 1 according to the present embodiment, blood pressure can be estimated based on pulse transit time Δt to continuously monitor a blood pressure for a long period of time while reducing a physical burden on a subject. Blood pressure estimation apparatus 1 can also perform both of the measurement of a blood pressure by the oscillometric method and the estimation of a blood pressure based on pulse transit time Δt, and accordingly can provide improved convenience and easily perform calibration between pulse transit time Δt and blood pressure.

In blood pressure estimation apparatus 1 according to the present embodiment, pulse wave detection unit 40E is disposed on the external surface of first fluid bag 21 for measuring blood pressure by the oscillometric method, and pulse wave detection unit 40E is pressed against palm lateral surface 90a of left wrist 90 upon expansion of first fluid bag 21, thereby detecting a pulse wave. A fluid supply unit can thus be used in common for the measurement of blood pressure by the oscillometric method and the detection of pulse wave, leading to a simplified configuration of blood pressure estimation apparatus 1.

Blood pressure estimation apparatus 1 according to the present embodiment can adjust, when at least one of first pulse wave detection unit 401 and second pulse wave detection unit 402 is located outside of the appropriate position range, a ratio between the volume of the fluid in first fluid bag 21 and the volume of the fluid in second fluid bag 22 to adjust the positions of first pulse wave detection unit 401 and second pulse wave detection unit 402 relative to radial artery 91 of left wrist 90 within the appropriate position range, thus estimating a blood pressure while guaranteeing reliability based on pulse transit time Δt. Also, a wide range in which the position of pulse wave detection unit 40E is adjustable can be secured, leading to stable blood pressure estimation.

Although first fluid bag 21 and second fluid bag 22 are used and pulse wave detection unit 40E is disposed on the external surface of first fluid bag 21 in the present embodiment, the present invention is not limited thereto. For example, each of first fluid bag 21 and second fluid bag 22 may be divided at a middle position in width direction Y. In this case, first pulse wave detection unit 401 and second pulse wave detection unit 402 are disposed on the external surfaces of separate fluid bags. Consequently, the positions of first pulse wave detection unit 401 and second pulse wave detection unit 402 can be adjusted separately.

Also, although a blood pressure is estimated based on pulse transit time Δt in the present embodiment, the present invention is not limited thereto. For example, a blood pressure may be estimated based on the waveform of pulse wave signal PS1 detected by first pulse wave detection unit 401. In this case, the position of first pulse wave detection unit 401 is adjusted such that the maximum amplitude value of pulse wave signal PS1 is more than or equal to a threshold.

It is noted that the embodiments disclosed herein are illustrative in every respect, and do not provide grounds for restrictive interpretation. Therefore, the technical scope of the present invention should not be interpreted by the above embodiments only, and is defined based on the description in the scope of the claims. Further, any modifications within the meaning and scope equivalent to the scope of the claims are encompassed.

What is claimed is:

1. A blood pressure estimation apparatus comprising:
a belt surrounding a measurement site;
a first fluid bag and a second fluid bag that are located side by side along an inner circumference of the belt, expand and contract upon entry and exit of a fluid, and are provided to press the measurement site from therearound while surrounding the measurement site;
a pulse wave sensor including a pulse wave detection unit that detects a pulse wave of an artery passing through the measurement site;
a fluid supply unit that supplies the fluid to the first fluid bag and the second fluid bag;
a first pressure sensor that detects a pressure in the first fluid bag; and
a second pressure sensor that detects a pressure in the second fluid bag, wherein
the pulse wave detection unit is disposed on an external surface portion of the first fluid bag and provided to press the measurement site upon expansion of the first fluid bag, and
a position of the pulse wave detection unit relative to the artery passing through the measurement site is adjusted through adjustment of a ratio between a volume of the fluid in the first fluid bag and a volume of the fluid in the second fluid bag by the fluid supply unit.

2. The blood pressure estimation apparatus according to claim 1, wherein the pulse wave detection unit detects a pulse wave based on a change in an impedance of the artery passing through the measurement site.

3. The blood pressure estimation apparatus according to claim 1, wherein the fluid supply unit includes
a pump that delivers the fluid,
a first on-off valve connected between the first fluid bag and the pump, and
a second on-off valve connected between the second fluid bag and the pump.

4. The blood pressure estimation apparatus according to claim 2, wherein the fluid supply unit includes
a pump that delivers the fluid,
a first on-off valve connected between the first fluid bag and the pump, and
a second on-off valve connected between the second fluid bag and the pump.
